(19) United States
(12) Patent Application Publication
(10) Pub. No.: US 2020/0179253 A1
(43) Pub. Date: Jun. 11, 2020

(54) NATURAL COSMETIC CLEANSER WHICH IS SOLID AT ROOM TEMPERATURE

(71) Applicant: Henkel AG & Co. KGaA, Duesseldorf (DE)

(72) Inventors: HEIKE SCHELGES, Willich (DE); ELVIRA SCHOLZ, Langenfeld (DE)

(73) Assignee: Henkel AG & Co. KGaA, Duesseldorf (DE)

(21) Appl. No.: 16/675,925

(22) Filed: Nov. 6, 2019

(30) Foreign Application Priority Data
Dec. 5, 2018 (DE) 10 2018 221 038.1

(51) Int. Cl. A61K 8/41; A61K 8/42; A61K 8/36; A61K 8/92; A61K 8/73; A61Q 19/10

(52) U.S. Cl. CPC A61K 8/416; A61K 8/42; A61Q 19/10; A61K 8/92; A61K 8/73; A61K 8/36

(57) ABSTRACT

The present disclosure provides a cosmetic cleanser that is solid at about 25° C., including—with respect to its weight—from about 5% to about 90% by weight of alkali salts of fatty acids (soaps); from about 2% to about 50% by weight of acyl glutamate(s); and from about 0.1% to about 10% by weight of betaine (N,N,N-trimethylammonioacetate or N,N,N-trimethylglycine).

### CROSS-REFERENCE TO RELATED APPLICATION

This application claims priority to German Patent Application No. 10 2018 221 038.1, filed Dec. 5, 2018, which is incorporated herein by reference in its entirety.

### TECHNICAL FIELD

The present disclosure relates to natural solid cleansers and conditioners for skin, and in particular for hair, which satisfy consumers' increasing desire for ecologically acceptable and natural products.

### BACKGROUND

Consumers are increasingly demanding cosmetic products which carry a natural cosmetics label and which are free from disputed ingredients. In order to provide the consumer with an assurance that they are buying a natural product which is ecologically impeccable and safe, a huge number of certifying enterprises grant an appropriate seal of approval following individual testing.

This includes, inter alia, the BDIH label as an identifier for products which satisfy the requirements for genuine natural cosmetics. Currently, about 200 license holders and over about 300 brands have been granted the BDIH label (Bundesverband der Industrie- and Handelsuntemehmen filr Arzneimittel, Reformwaren, Nahrungserganzungs- and kosmetische Mittel, (Federal Association of German Industrial and Commercial Enterprises for Pharmaceuticals, Health Products, Food Supplements and Cosmetics), abbreviated to BDIH). Approximately 40 are located in Europe outside Germany and a further about 40 are outside Europe. In total, there are currently approximately 9000 products which have been awarded the BDIH seal.

As a supplement to the BDIH seal for controlled natural cosmetics, other seals have been established, for example the French “Ecocert” label. “Organic” has the same requirements for ingredients as another seal, the “NaTrue seal”. This is the same for “Made with organic ingredients”. For American products, there is the “USDA seal”, and in England there is also the “Soil Association” label.

Since 2010, a further seal with the name “Cosmos” has been becoming established for natural cosmetics and biocosmetics. The new label is binding for all innovations in natural cosmetics and biocosmetics. It covers all members of BDIH, Cosmebio, ICEA, Ecocert and the Soil Association. Overall, they represent the largest global system for the certification of bio-beauty products. Since that time, products from the members have been certified in accordance with the international Cosmos standard AISBL. Several thousand raw materials and also products have now been successfully tested. All results for natural cosmetics or biocosmetics which have been filed and tested since Jan. 1, 2017 then obtain the Cosmos certification from the respective member organization. Thus, there is the Cosmos-BDIH label or the Cosmos-Ecocert label, for example.

A distinction is made between the inspection of natural cosmetics and the certification of bioproducts. The latter must additionally demonstrate that specified proportions of the ingredients are of biological origin. Thus, for a biocosmetic product, at least about 95% of the components obtained from plants must derive from an ecological agricultural source.

Furthermore, there is a growing trend for products which can dispense with plastic outer packaging. In this case, systems which are low in water such as soaps and syndets come to the fore. In order to be able to guarantee both good performance on skin and hair along with simultaneous mildness, only a few foaming surfactants in powder form are available which also correspond to the Cosmos standard.

There is therefore a need for cosmetic cleansers and conditioners for skin, and in particular for hair, which are Cosmos certifiable and which can be marketed with a significantly reduced amount of packaging.

### BRIEF SUMMARY

The present disclosure addresses at least some of the problems mentioned above. The present disclosure is in part characterized by employing, in a first aspect, a cosmetic cleanser which is solid at about 25° C., containing—respectively with respect to its weight—

a) from about 5% to about 90% by weight of alkali salts of fatty acids (soaps);
b) from about 2% to about 50% by weight of acyl glutamate(s);
c) from about 0.1% to about 10% by weight of betaine (N,N,N-trimethylammonioacetate or N,N,N-trimethylglycine).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The cleansers as contemplated herein are solid at about 25° C. “Solid materials” in the context of the present application means three-dimensional structures which have a stable shape, which are not liquid or gaseous, i.e. retain their external shape even without a vessel surrounding it. However, the term “solid” is silent as regards the density or elasticity or other physical properties, so that gels, jellies, butters etc. may also be considered to be “solid” within the meaning of the present disclosure, as long as they are stable in shape at about 25° C.

As the first ingredient, the cleansers as contemplated herein contain one or more soap(s) in a total quantity of all alkali salts of fatty acids of from about 5% to about 90% by weight. Particularly, the soap(s) are used within narrow ranges of quantities. In this case, cosmetic cleansers as contemplated herein contain from about 10% to about 89% by weight, for example from about 20% to about 88% by weight, such as from about 30% to about 87% by weight, such as from about 40% to about 86% by weight and in particular from about 50% to about 85% by weight of alkali salts of fatty acids (soaps).

Fatty acids are aliphatic carboxylic acids with the formula R¹CO—OH, in which R¹CO represents an aliphatic, linear or branched acyl residue containing 6 to 22 carbon atoms and 0 and/or 1, 2 or 3 double bonds. Some examples are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucaic acid, as well as their technical mixtures which, for example, precipitate out during the hydrolysis of natural fats and oils under pressure. Technical fatty acids containing 12 to 18 carbon atoms such as, for example, coconut, palm, palm kernel or tallow fatty acid, are suitable, wherein having regard to the present disclosure, the use of coconut fatty acids is exemplary and the use of raw materials from palm oil should be dispensed with.

Exemplary cleansers as contemplated herein are free from soaps which are produced from palm oil.

Sodium salts are exemplary alkali salts because of their application-related properties in the context of the present disclosure. Suitable cleansers as contemplated herein contain from about 10% to about 89% by weight, for example from about 20% to about 88% by weight, such as from about 30% to about 87% by weight, such as from about 40% to about 86% by weight and in particular from about 50% to about 85% by weight of sodium salts of C₁₂₋₁₈ fatty acids.

As the second ingredient, the cleansers as contemplated herein contain from about 2% to about 50% by weight of acyl glutamate(s). These compounds are produced from L-glutamic acid and fatty acids which can be found in nature and are exemplified by low foam formation and good washing properties.

In an embodiment, the cosmetic cleansers as contemplated herein contain from about 3% to about 40% by weight, for example from about 4% to about 35% by weight, such as from about 5% to about 30% by weight, such as from about 6% to about 25% by weight and in particular from about 7.5% to about 15% by weight of acyl glutamates with formula (I)

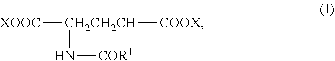

in which RICO represents a linear or branched acyl residue containing 6 to 22 carbon atoms and 0 and/or 1, 2 or 3 double bonds and X represents hydrogen, an alkali and/or alkaline-earth metal, ammonium, alkylammonium, alkanolammonium or glucammonium.

Again with a view to the possibility of certification as a natural cosmetic, in the context of the present disclosure, it is exemplary to use alkali metal ions and in particular sodium ions as the cation X. Regarding the fatty residues, as was the case with the soaps, the use of coconut fatty acids is suitable, and the use of raw materials from palm oil should be dispensed with.

Exemplary cleansers as contemplated herein are free from acyl glutamates which are produced from palm oil.

More exemplary cosmetic cleansers as contemplated herein contain an acyl glutamate in which X represents Na and R¹CO represents an acyl residue which is derived from coconut oil (INCI: Sodium Cocoyl Glutamate).

In addition to the soap(s) and the acyl glutamate(s), the cleansers as contemplated herein may additionally contain further surfactants. Having regard to the problems mentioned above and the compatibility of the cleansers with the skin, it has been shown to be advantageous if only surfactants from specific groups of materials are used. Having regard to consumer acceptance, skin compatibility and application properties, cosmetic cleansers as contemplated herein which exclusively contain surfactants from the groups formed by:
alkyl oligosaccharides and polysaccharides
betaines
   are suitable.

In an embodiment, the cosmetic cleansers as contemplated herein—with respect to their weight—contain less than about 1% by weight, for example less than about 0.5% by weight, such as less than about 0.5% by weight and in particular less than about 0.1% by weight of sulfate-containing surfactant(s), wherein some cleansers are completely free from sulfate-containing surfactants.

The optional surfactants which may be used are described below.

Alkyl and alkenyl oligoglycosides constitute known non-ionic surfactants which can be described by the formula

R¹O-[G]ₚ

in which R¹ represents an alkyl and/or alkenyl residue containing 4 to 22 carbon atoms, G represents a sugar residue containing 5 or 6 carbon atoms and p represents numbers from about 1 to about 10. They can be obtained using relevant preparative organic chemistry methods.

The alkyl oligoglycosides and/or alkenyl oligoglycosides may be derived from aldoses or ketoses containing 5 or 6 carbon atoms, for example from glucose. Suitable alkyl oligoglycosides and/or alkenyl oligoglycosides are thus alkyl- and/or alkenyl oligoglucosides. The index number p in the general formula gives the degree of oligomerization (DP), i.e. the distribution of monoglycosides and oligoglycosides, and represents a number between about 1 and about 10. While p must always be a whole number in the compound given, and above all can take the value p=from about 1 to about 6 in this case, the value p for a specific alkyl oligoglycoside is an analytically determined mathematical factor which usually represents a fractional number. For example, alkyl oligoglycosides and/or alkenyl oligoglycosides with a mean degree of oligomerization p of from about 1.1 to about 3.0 are used. From an application-related viewpoint, such alkyl oligoglycosides and/or alkenyl oligoglycosides are suitable which have a degree of oligomerization of less than about 1.7 and in particular between about 1.2 and about 1.4. The alkyl residue or alkenyl residue R¹ may be derived from primary alcohols containing 4 to 11, for example 8 to 10 carbon atoms. Some examples are butanol, caproic alcohol, caprylic alcohol, capric alcohol and undecyl alcohol as well as their technical mixtures as obtained, for example, by the hydrolysis of technical fatty acid methyl esters or during the hydrolysis of aldehydes in the Roelen oxosynthesis process. Alkyl oligoglucosides with a chain length of C₈-C₁₀ (DP=1 to 3), which arise as a precursor in the distillative separation of technical C₈-C₁₈ coconut oil alcohol and which may be contaminated with a proportion of less than 6% by weight of C₁₂ alcohol, as well as alkyl oligoglucosides based on technical C_{9/11} oxo alcohols (DP=from about 1 to about 3), are suitable. The alkyl or alkenyl residue R¹ may also be derived from primary alcohols containing 12 to 22, for example 12 to 14 carbon atoms. Some examples are lauryl alcohol, myristyl alcohol, cetyl alcohol, palmoleyl alcohol, stearyl alcohol, isostearyl alcohol, oleyl alcohol, elaidyl alcohol, petroselinic alcohol, arachidyl alcohol, gadoleyl alcohol, behenyl alcohol, erucyl alcohol, brassidyl alcohol as well as their technical mixtures, which may be obtained as described above. Alkyl oligoglucosides based on hardened C_{12/14} coconut alcohol with a DP of from about 1 to about 3 are suitable.

Exemplary cleansers as contemplated herein contain from about 0.1% to about 10% by weight, for example from about 0.25% to about 7.5% by weight, such as from about 0.5% to about 5% by weight, such as from about 0.75% to about 2.5% by weight and in particular from about 1% to about 2% by weight of surfactant(s) from the group formed by alkyl oligosaccharides and alkyl polysaccharides.

Betaines are known surfactants which may be produced by carboxyalkylation, for example carboxymethylation of amine compounds. For example, the starting materials are condensed with halocarboxylic acids or their salts, in particular with sodium chloroacetate, wherein one mole of salt is formed per mole of betaine. Furthermore, the deposition of unsaturated carboxylic acids such as acrylic acid, for example, is also possible. Examples of suitable betaines are the carboxyalkylation products of secondary amines and in particular tertiary amines, which may be described by the formula:

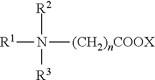

in which R¹ represents alkyl residues and/or alkenyl residues containing 6 to 22 carbon atoms, R² represents hydrogen or alkyl residues containing 1 to 4 carbon atoms, R³ represents alkyl residues containing 1 to 4 carbon atoms, n represents numbers from about 1 to about 6 and X represents an alkali and/or alkaline-earth metal or ammonium. Typical examples are carboxymethylation products of hexylmethylamine, hexyldimethylamine, octyldimethylamine, decyldimethylamine, dodecylmethylamine, dodecyldimethylamine, dodecylethylmethylamine, C_{12/14} cocoalkyldimethylamine, myristyldimethylamine, cetyldimethylamine, stearyldimethylamine, stearylethylmethylamine, oleyldimethylamine, C_{16/18} tallow alkyldimethylamine as well as their technical mixtures.

Furthermore, carboxyalkylation products of amidoamines described by the following formula:

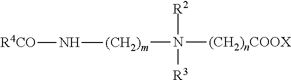

may also be considered, in which R⁴CO represents an aliphatic acyl residue containing 6 to 22 carbon atoms and 0 or from about 1 to about 3 double bonds, m represents numbers from 1 to 3 and R², R³, n and X have the meanings given above. Typical examples are reaction products of fatty acids containing 6 to 22 carbon atoms, namely caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucaic acid as well as their technical mixtures, with N,N-dimethylaminoethylamine, N,N-dimethylaminopropylamine, N,N-diethylaminoethylamine and N,N-diethylaminopropylamine, which are condensed with sodium chloroacetate. For example, a condensation product of C_{8/18} coconut fatty acid-N,N-dimethylaminopropylamide with sodium chloroacetate is used.

Furthermore, suitable starting materials for the betaines used in the context of the present disclosure which may also be considered are imidazolines, which can be described by the formula:

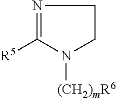

in which R⁵ represents an alkyl residue containing 5 to 21 carbon atoms, R⁶ represents a hydroxy group, an OCOR⁵ or NHCOR⁵ residue and m represents 2 or 3. These substances too are known substances which, for example, may be obtained by cyclization condensation of 1 or 2 moles of fatty acid with multivalent amines such as, for example, aminoethyl ethanolamine (AEEA) or diethylene triamine. The corresponding carboxyalkylation products constitute mixtures of different open-chained betaines. Typical examples are condensation products of the fatty acids cited above with AEEA, for example imidazolines based on lauric acid or, in fact, C_{12/14} coconut fatty acid, which are then turned into betaines with sodium chloroacetate.

Exemplary cleansers as contemplated herein contain from about 0.5% to about 15% by weight, for example from about 1% to about 10% by weight, such as from about 1.5% to about 7.5% by weight, such as from about 2% to about 6% by weight and in particular from about 2.5% to about 5% by weight of surfactant(s) from the betaine group.

As the third ingredient, the cleansers as contemplated herein contain from about 0.1% to about 10% by weight of betaine (N,N,N-trimethylammonioacetate or N,N,N-trimethylglycine). For example, betaine is used both within narrow ranges of quantities and also from native sources. Herein, cosmetic cleansers as contemplated herein which contain from about 0.1% to about 10% by weight, for example from about 0.2% to about 9% by weight, such as from about 0.3% to about 8% by weight, such as from about 0.4% to about 7% by weight, such as from about 0.45% to about 6% by weight and in particular from about 0.5% to about 5% by weight of natural betaine are suitable.

As a further ingredient, the substances as contemplated herein may contain at least one natural polymer which has not been chemically modified. Substances of this type, also termed biopolymers, derive in particular from the polysaccharide group. The use of cellulose, starch, guar gum or xanthan is suitable.

The term “which has not been chemically modified” means that the natural polymer has not undergone any chemical reactions in order to modify its properties. In contrast, “physical modifications” are possible, and are common with starches and xanthan, for example (pregelatinization, cooking, cold-swelling or instantized starches, heat-treated xanthan). Physically modified starches are equated with native starches because they have only been heat-treated, i.e. cooked.

In an embodiment, the substances as contemplated herein contain from about 0.1% to about 5% by weight of guaran (INCI name Guar Gum).

Guaran, also known as guar gum, is a vegetable gum. The chemical bond from the polysaccharide group is the major component of guar germ meal (or guar meal for short). Guaran includes D-mannopyranose units which are bonded together via β-glycosidic bonds. In addition, alternate mannopyranose units carry α-D-galactopyranosyl residues via a bond.

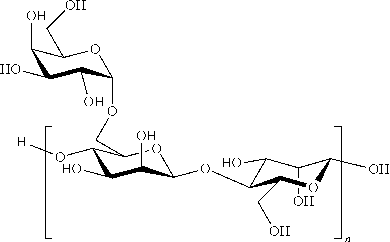

For example, the guar gum is used in narrower ranges of quantities. Here, cleansers as contemplated herein contain from about 0.2% to about 4% by weight, for example from about 0.3% to about 3.5% by weight, such as from about 0.4% to about 3% by weight, such as from about 0.45% to about 2.5% by weight and in particular from about 0.5% to about 2% by weight of guaran.

When they contain a biopolymer, the substances as contemplated herein may contain from about 0.1% to about 5% by weight of xanthan (INCI name Xanthan Gum). Xanthan gum is a natural, sustainable raw material and is excreted as an anionic polysaccharide from the bacterium Xanthomonas campestris.

The molecular weight of the xanthan gum used may be about 2×10⁶ to 20×10⁶ g/mol.

Xanthan gum contains D-glucose, D-mannose, D-glucuronic acid, acetate and pyruvate as molecular components in an approximate molar ratio of about 28 to about 30 to about 20 to about 17 to about 5.1 to about 6.3. The polymeric backbone of the xanthan gum is formed by a cellulose chain formed from β-1,4-bonded glucose units. Xanthan contains structural units with the following formula:

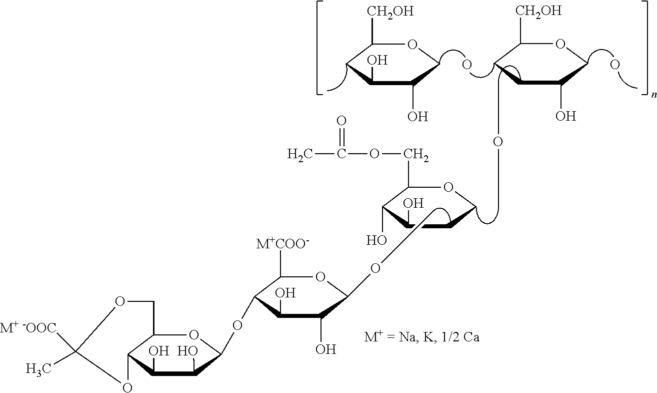

If appropriate, heat-treated xanthan gum may also be used in the cosmetic cleansers as contemplated herein.

In an embodiment, the cleanser as contemplated herein contains, as the xanthan gum:
heat-treated xanthan gum, or
a mixture of xanthan gum and heat-treated xanthan gum.

When using a mixture of xanthan gum and heat-treated xanthan gum, it has been shown to be effective for the xanthan gum and the heat-treated xanthan gum to be used in a weight ratio range of from about 2 to about 1 to about 1 to about 20, in particular of from about 1 to about 2 to about 1 to about 10.

The term “heat-treated xanthan gum” as used as contemplated herein should be understood to mean xanthan gum which is subjected to at least about 40° C. of heat. The resulting heat-treated xanthan gum has an improved dispersibility and can be dispersed faster in water than xanthan gum which has not undergone heat treatment. In a about 1% by weight aqueous solution, a suitable heat-treated xanthan gum has a viscosity of at least from about 25000 to about 40000 mPa·s (Brookfield DV-I viscosimeter, spindle #6 at about 23° C. and about 10 rpm). Suitable heat-treated xanthan gums which may be used produce a pH of from about 4.0 to about 6.0 at 23° C. when in a about 1% by weight aqueous solution.

The heat-treated xanthan gum which is exemplary as contemplated herein was obtained by tempering xanthan gum at a temperature of at least about 60° C., in particular at least about 100° C. Tempering may be carried out using a multitude of known methods such as, for example, by heat treatment using an oven, fluidized bed, infrared or microwave. In the context of the above heat treatments, it is furthermore suitable for the xanthan gum to have a water content of less than about 25% by weight, in particular of less than about 8% by weight, for example of less than about 3% by weight prior to the heat treatment. In an example as contemplated herein, the cleanser as contemplated herein should have therein such heat-treated xanthan gum which has been obtained by heat treating xanthan gum with a water content of less than about 25% by weight at a temperature of at least about 60° C. (in particular at least about 100° C.) for at least about 30 minutes. In an embodiment as contemplated herein, the cleanser as contemplated herein may employ such heat-treated xanthan gum wherein xanthan gum with a water content of less than about 8% by weight has been heat-treated at a temperature of at least about 60° C. (in particular at least about 100° C.) for at least about 30 minutes. An exemplary duration of said heat treatments of the xanthan gum—in particular with said water content—at a temperature of at least about 60° C. (in particular at least about 100° C.) is at least about 1 hour. Another exemplary duration of said heat treatments of the xanthan gum—in particular with a suitable water content—at a temperature of at least about 60° C. (in particular at least about 100° C.) is at least about 2.5 hours.

In an embodiment—irrespective of whether the xanthan gum is heat-treated xanthan or non-heat-treated xanthan or comprises a mixture of both—it is used in a narrow range of quantities. In this case, cleansers as contemplated herein contain from about 0.1% to about 10% by weight, for example from about 0.25% to about 9% by weight, such as from about 0.5% to about 8% by weight, such as from about 0.75% to about 7% by weight and in particular from about 1% to about 6% by weight of xanthan.

The substances as contemplated herein may be formulated so as to be low in water. This on the one hand provides the solid form of presentation, and on the other hand avoids unnecessary product volume, which would result in higher packaging and transport costs. Suitable cosmetic cleansers as contemplated herein contain less than about 25% by weight, for example less than about 20% by weight, such as less than about 15% by weight, such as less than about 10% by weight and in particular from about 0.1 to about 5% by weight of water.

In an embodiment, the substances as contemplated herein may contain further components, wherein, natural skin care and moisturizing substances are suitable. In an embodiment, native oils are used, so that cosmetic cleansers contain from about 0.1% to about 10% by weight, for example from about 0.25% to about 9% by weight, such as from about 0.5% to about 8% by weight, such as from about 0.75% to about 7% by weight and in particular from about 1% to about 6% by weight of natural oil(s).

In an embodiment, the cleansers as contemplated herein contain one or more of the following cited oils in a total oil quantity as defined above: acacia oil, algae oil, argan oil (from the fruit of the argan tree), avocado oil (from the flesh of the avocado fruit from the avocado tree), babassu oil, cottonseed oil (from the seeds of the cotton plant), borage oil or borage seed oil (from the seeds of the borage plant), cupuacu butter, cashew shell oil, thistle oil (also known as “safflower oil”, from the seeds of the safflower or carthamus), peanut oil (from the fruit of the peanut plant), hazelnut oil (from hazelnuts from the hazelnut bush), hemp oil (from the seeds of edible hemp), jatropha oil (from the seeds of jatropha curcas), jojoba oil (actually a liquid wax; from the seeds of the jojoba shrub), camomile oil (from the seeds of Camellia oleifera, Camellia sinensis or Camellia japonica), cocoa butter, coconut oil (from the seeds of the coconut, the fruit of the coconut palm tree), pumpkin seed oil (also known as pepita oil; from the seeds of the Styrian oil squash), linseed oil (from ripe linseed from flax), camelina oil (from the seeds of gold-of-pleasure, Brassicaceae family), macadamia oil (from the nuts of the macadamia tree), corn oil (from corn seeds), almond oil (from almonds from the almond tree), mango butter (from Mangifera indica), apricot kernel oil (from the apricot kernel—i.e. the pit of the apricot kernel—from the apricot), poppyseed oil (from the seeds of the poppy), evening primrose oil, olive oil (from the fruit and kernel of the olive, the fruit from the olive tree), palm oil (from the fruit of the palm fruit, the fruit of the oil palm), palm kernel oil (from the kernel of the palm fruit, the fruit of the oil palm), papaya oil, pistachio oil, pecan nut oil, perilla oil from the seeds of the perilla plant (shiso, Korean perilla), rapeseed oil (from the seeds from rape, Brassicaceae family), rice oil, castor oil (from the seed of the miracle tree), sallow thorn oil (from the fruit of sallow thorn berries, the fruit from the sallow thorn bush), sallow thorn kernel oil (from the kernels from sallow thorn berries, the fruit from the sallow thorn bush), mustard oil (from black mustard seeds), black cumin oil (from the seeds of the fruit capsule from the black cumin plant), sesame oil (from the seeds of the sesame plant), shea butter (from the seeds of the shea nut tree), soya oil (from soybeans), sunflower oil (from sunflower seeds), tung oil, walnut oil (from the kernels from the nuts from the walnut tree), watermelon seed oil, grapeseed oil (from the kernels from the fruit (grapes) of the grape vine or vine), wheatgerm oil (from wheatgerm), and cedarwood oil (from the wood from cedar of Lebanon).

The substances as contemplated herein may contain further ingredients; within the spirit and scope of the present disclosure, care should be taken that COSMOS certified raw materials are employed.

### Examples

The following compositions may be produced by way of example (all details are as a % by weight):

| | 01 | 02 | 03 | 04 | 05 | 06 | 07 | 08 | 09 |
|---|---|---|---|---|---|---|---|---|---|
| Sodium Sunflowerseedate | 60.86 | 60.86 | 60.86 | 60.86 | 60.86 | 60.86 | 60.86 | 60.86 | 60.86 |
| Sodium Cocoate | 12.96 | 12.96 | 12.96 | 12.96 | 12.96 | 12.96 | 12.96 | 12.96 | 12.96 |
| Sodium Cocoyl | 5.00 | 5.00 | 5.00 | 7.00 | 3.00 | 5.00 | 5.00 | 5.00 | 5.00 |
| Glutamate | | | | | | | | | |
| Cocamidopropyl Betaine | 4.19 | 4.19 | 4.19 | 3.00 | 7.00 | 4.19 | 4.19 | 4.19 | 4.19 |
| Glycerin | 2.59 | 2.59 | 2.59 | 2.59 | 2.59 | 2.59 | 2.59 | 2.59 | 2.59 |
| Cocos Nucifera | 2.00 | 2.00 | 2.00 | 2.00 | 2.00 | 3.00 | 5.00 | 3.00 | 3.00 |
| (Coconut) Oil | | | | | | | | | |
| Sodium Chloride | 1.60 | 1.60 | 1.60 | 1.60 | 1.60 | 1.60 | 1.60 | 1.60 | 1.60 |
| Citric Acid | 0.43 | 0.43 | 0.43 | 0.43 | 0.43 | 0.43 | 0.43 | 0.43 | 0.43 |
| Betaine | 0.15 | 1.00 | 0.50 | 0.15 | 0.09 | 0.20 | 0.50 | 1.00 | 2.00 |
| Tetrasodium Glutamate | 0.05 | 0.05 | 0.05 | 0.05 | 0.05 | 0.05 | 0.05 | 0.05 | 0.05 |
| Diacetate | | | | | | | | | |
| Parfum (Fragrance) | 1.00 | 1.00 | 1.00 | 1.00 | 1.00 | 1.00 | 1.00 | 1.00 | 1.00 |

| | |
|---|---|
| Aqua (Water, Eau) | ad 100 |

| | 10 | 11 | 12 | 13 | 14 | 15 | 16 | 17 | 18 |
|---|---|---|---|---|---|---|---|---|---|
| Sodium Palmate | 57.08 | 56.49 | 56.49 | 56.49 | 56.49 | 56.49 | 56.49 | 56.49 | 56.49 |
| Sodium Palm Kemelate | 14.51 | 14.36 | 14.36 | 14.36 | 14.36 | 14.36 | 14.36 | 14.36 | 14.36 |
| Sodium Cocoyl | 5.00 | 5.00 | 5.00 | 5.00 | 7.00 | 3.00 | 5.00 | 5.00 | 5.00 |
| Glutamate | | | | | | | | | |
| Cocamidopropyl Betaine | 4.19 | 4.19 | 4.19 | 4.19 | 3.00 | 7.00 | 4.19 | 4.19 | 4.19 |
| Glycerin | 4.00 | 4.00 | 4.00 | 4.00 | 4.00 | 4.00 | 4.00 | 4.00 | 4.00 |
| Cocos Nucifera | 2.00 | 2.00 | 2.00 | 2.00 | 2.00 | 3.00 | 5.00 | 3.00 | 3.00 |
| (Coconut) Oil | | | | | | | | | |
| Sodium Chloride | 1.21 | 1.59 | 1.59 | 1.59 | 1.59 | 1.59 | 1.59 | 1.59 | 1.59 |
| Sodium Hydroxide | 0.04 | 0.04 | 0.04 | 0.04 | 0.04 | 0.04 | 0.04 | 0.04 | 0.04 |
| Betaine | 0.10 | 1.00 | 0.50 | 0.20 | 0.50 | 0.15 | 0.10 | 1.00 | 2.00 |
| Tetrasodium Glutamate | 0.05 | 0.05 | 0.05 | 0.05 | 0.05 | 0.05 | 0.05 | 0.05 | 0.05 |
| Diacetate | | | | | | | | | |
| Parfum (Fragrance) | 1.00 | 1.00 | 1.00 | 1.00 | 1.00 | 1.00 | 1.00 | 1.00 | 1.00 |

| | |
|---|---|
| Aqua (Water, Eau) | ad 100 |

| | 19 | 20 | 21 | 22 | 23 | 24 | 25 | 26 | 27 |
|---|---|---|---|---|---|---|---|---|---|
| Potassium Olivate | 75.98 | 75.98 | 75.98 | 75.98 | 75.98 | 75.98 | 75.98 | 75.98 | 75.98 |
| Sodium Cocoyl | 5.00 | 5.00 | 5.00 | 7.00 | 3.00 | 5.00 | 5.00 | 5.00 | 5.00 |
| Glutamate | | | | | | | | | |
| Cocamidopropyl Betaine | 4.19 | 4.19 | 4.19 | 3.00 | 7.00 | 4.19 | 4.19 | 4.19 | 4.19 |
| Cocos Nucifera | 2.00 | 2.00 | 2.00 | 2.00 | 2.00 | 3.00 | 5.00 | 3.00 | 3.00 |
| (Coconut) Oil | | | | | | | | | |
| Sodium Chloride | 1.60 | 1.60 | 1.60 | 1.60 | 1.60 | 1.60 | 1.60 | 1.60 | 1.60 |
| Glycerin | 0.86 | 0.86 | 0.86 | 0.86 | 0.86 | 0.86 | 0.86 | 0.86 | 0.86 |
| Betaine | 0.15 | 1.00 | 0.50 | 0.20 | 0.50 | 1.50 | 0.10 | 1.00 | 2.00 |
| Tetrasodium Glutamate | 0.05 | 0.05 | 0.05 | 0.05 | 0.05 | 0.05 | 0.05 | 0.05 | 0.05 |
| Diacetate | | | | | | | | | |
| Parfum (Fragrance) | 1.00 | 1.00 | 1.00 | 1.00 | 1.00 | 1.00 | 1.00 | 1.00 | 1.00 |

| | |
|---|---|
| Aqua (Water, Eau) | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic cleanser which is solid at about 25° C. comprising, respectively with respect to its weight,
a) from about 5% to about 90% by weight of alkali salts of fatty acids (soaps);
b) from about 2% to about 50% by weight of acyl glutamate(s);
c) from about 0.1% to about 10% by weight of betaine (N,N,N-trimethylammonioacetate or N,N,N-trimethylglycine).

2. The cosmetic cleanser as claimed in claim 1, wherein it comprises from about 10% to about 89% by weight of alkali salts of fatty acids (soaps).

3. The cosmetic cleanser as claimed in claim 1, wherein it comprises from about 10% to about 89% by weight of sodium salts of C₂₋₁₈ fatty acids.

4. The cosmetic cleanser as claimed in claim 1, wherein it comprises from about 3% to about 40% by weight of acyl glutamates with formula (I) 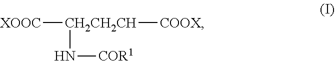
in which R¹CO represents a linear or branched acyl residue comprising 6 to 22 carbon atoms and 0 and/or 1, 2 or 3 double bonds and X represents hydrogen, an alkali and/or alkaline-earth metal, ammonium, alkylammonium, alkanolammonium or glucammonium.

5. The cosmetic cleanser as claimed in claim 4, wherein it comprises an acyl glutamate in which X represents Na and R¹CO represents an acyl residue which is derived from coconut oil (INCI: Sodium Cocoyl Glutamate).

6. The cosmetic cleanser as claimed in claim 1, wherein it comprises from about 0.1% to about 10% by weight of natural betaine.

7. The cosmetic cleanser as claimed in claim 1, wherein it comprises from about 0.1% to about 10% by weight of xanthan.

8. The cosmetic cleanser as claimed in claim 1, wherein it comprises less than about 25% by weight of water.

9. The cosmetic cleanser as claimed in claim 1, wherein it comprises from about 0.1% to about 10% by weight of natural oil(s).

10. The cosmetic cleanser as claimed in claim 1, wherein it comprises—with respect to its weight—less than about 1% by weight of sulfate-containing surfactant(s).

11. The cosmetic cleanser as claimed in claim 1, wherein it comprises from about 20% to about 88% by weight of alkali salts of fatty acids (soaps).

12. The cosmetic cleanser as claimed in claim 1, wherein it comprises from about 20% to about 88% by weight of sodium salts of C₁₂₋₁₈ fatty acids.

13. The cosmetic cleanser as claimed in claim 1, wherein it comprises from about 4% to about 35% by weight of acyl glutamates with formula (I) 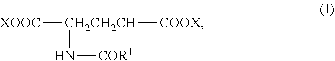
in which R¹CO represents a linear or branched acyl residue comprising 6 to 22 carbon atoms and 0 and/or 1, 2 or 3 double bonds and X represents hydrogen, an alkali and/or alkaline-earth metal, ammonium, alkylammonium, alkanolammonium or glucammonium.

14. The cosmetic cleanser as claimed in claim 1, wherein it comprises from about 0.2% to about 9% by weight of natural betaine.

15. The cosmetic cleanser as claimed in claim 1, wherein it comprises from about 0.25% to about 9% by weight of xanthan.

16. The cosmetic cleanser as claimed in claim 1, wherein it comprises less than about 20% by weight of water.

17. The cosmetic cleanser as claimed in claim 1, wherein it comprises from about 0.25% to about 9% by weight of natural oil(s).

18. The cosmetic cleanser as claimed in claim 1, wherein it comprises—with respect to its weight—less than about 0.5% by weight of sulfate-containing surfactant(s).

19. The cosmetic cleanser as claimed in claim 1, wherein it comprises less than about 15% by weight of water.

20. The cosmetic cleanser as claimed in claim 1, wherein it comprises less than about 10% by weight of water.
